(19) United States
(12) Patent Application Publication
(10) Pub. No.: US 2025/0359761 A1
(43) Pub. Date: Nov. 27, 2025

(54) NEUROLOGICAL CONDITION CHARACTERIZATION AND DIAGNOSIS SYSTEMS, DEVICES, AND METHODS

(71) Applicant: THE BOARD OF REGENTS OF THE UNIVERSITY OF TEXAS SYSTEM, Austin, TX (US)

(72) Inventor: Brett Anthony Whittemore, Dallas, TX (US)

(21) Appl. No.: 18/872,733

(22) PCT Filed: Jun. 6, 2023
(86) PCT No.: PCT/US2023/068005

§ 371 Date: Dec. 6, 2024

Related U.S. Application Data
(60) Provisional application No. 63/349,650, filed on Jun. 7, 2022.

(51) Int. Cl. A61B 5/00; A61B 5/03; A61B 5/08; A61B 5/37

(52) U.S. Cl. CPC A61B 5/0075; A61B 5/031; A61B 5/0816; A61B 5/37; A61B 5/4806; A61B 5/7282; A61B 5/742

(57) ABSTRACT

Systems, methods, and devices include a hydrocephalus characterizing device for collecting data to detect an abnormal neurological condition. The hydrocephalus characterizing device includes one or more sensors such as a first sensor and a second sensor. The first sensor is an optical sensor (e.g., a near infrared spectroscopy sensor), and the second sensor is a biometric sensor (e.g., a photoplethysmography sensor). The hydrocephalus characterizing device also includes a memory storing instructions that, when executed by a processor, cause the hydrocephalus characterizing device to receive first data generated by the first data, the first data representing intracranial waveforms. The instructions also cause the hydrocephalus characterizing device to receive second data, from the second sensor, the second data representing a non-cranial biophysical characteristic. An intracranial pulse state is determined by the hydrocephalus characterizing device based on the first data and the second data, from which an abnormal neurological characteristic is detected.

### CROSS-REFERENCE TO RELATED APPLICATION

This application claims priority to U.S. Provisional Patent Application No. 63/349,650, filed Jun. 7, 2022 and titled “NEUROLOGICAL CONDITION CHARACTERIZATION AND DIAGNOSIS SYSTEMS, DEVICES, AND METHODS,” the entirety of which is herein incorporated by reference.

### BACKGROUND

### 1. Field

The present disclosure relates generally to the field of brain imaging and, more particularly, to neurological condition diagnostics systems, methods, and devices.

### 2. Discussion of Related Art

Hydrocephalus, the abnormal buildup of cerebrospinal fluid (CSF), affects 1 in 1000 people. It is typically treated with an implanted tube called a shunt to divert CSF to another space in the body, or with an endoscopic third ventriculostomy (ETV), where a CSF bypass pathway is created within the brain. These surgical treatments fail often. For example, a shunt has at least a 50% likelihood of requiring revision surgery, and ETVs have a similar failure rate depending on the underlying cause of hydrocephalus. Shunt failure, if not detected and treated in a timely manner, can result in brain damage or death.

The typical clinical pathway for diagnosing surgical failure is as follows. A patient develops symptoms such as headaches, nausea, vomiting, lethargy, cognitive decline, or coma. A CT or MRI scan is performed to assess accumulation of CSF by looking for an increase in the size of the ventricles (CSF chambers in the brain), and the shunt is tapped with a needle to evaluate flow. This process often results in a diagnostic dilemma. Many of the above symptoms are not highly specific, and when the CT or MRI is misleading, which occurs about 30% of the time, the decision for reoperation is often based on clinical judgment and surgeon philosophy, resulting in a wide variation in practices.

One technique to evaluate shunt malfunction involves using noninvasive devices for detecting the CSF flow through the shunt. These devices are based on a thermoconvective mechanism: a temperature sensor is placed on the skin over the shunt tubing where it crosses the clavicle, and the skin temperature over the shunt tubing is changed upstream of the sensor (by cooling or warming the skin) so that CSF flow through the shunt is detected as a temperature change. However, shunt flow has been proven to be intermittent. If flow is not confirmed by the device, the shunt system can still be functional, or flow may be detected but inadequate for the individual patient. The low sensitivity of shunt flow detection for determining shunt malfunction and determining the need to operate limits the usefulness of this technique. Rather than directly addressing the intracranial environment, these thermoconvective devices assess shunt flow, which can be difficult to interpret when making a decision about whether to perform a shunt revision operation.

Better diagnostic tools are needed. It is with these observations in mind, among others, that various aspects of the present disclosure were conceived and developed.

### BRIEF SUMMARY

To address the foregoing problems, the presently disclosed technology can provide systems, methods, and devices to characterize a neurological condition. For instance, a method can include positioning a first sensor at a first location communicatively coupled to a skull of a patient, the first sensor being a near infrared spectroscopy (NIRS) sensor; and/or positioning a second sensor at a second location communicatively coupled to a peripheral neuropathy of the patient, the second sensor being a biometric sensor. The method can also include receiving first data representing intracranial waveforms generated by intracranial pulsatility detected by the first sensor; receiving second data representing a non-cranial biophysical characteristic detected by the second sensor; and/or determining an intracranial pulsatile state of the patient based on the first data and the second data. Furthermore, the method can include detecting an abnormal neurological characteristic based on the intracranial pulsatile state; and/or generating an output representing the abnormal neurological characteristic at a display of a clinic device.

In some examples, the method further includes performing a spectral analysis on the first data to determine one or more beat-to-beat waveform signatures representing intracranial activity. The one or more beat-to-beat waveform signatures can be correlated to the non-cranial biophysical characteristic to detect the abnormal neurological characteristic. Additionally, detecting of the abnormal neurological characteristic can include comparing the intracranial pulsatile state of the patient to a reference intracranial pulsatile state based on previously generated data. The method can further include determining a first frequency band corresponding to a cardiac process; and/or determining a second frequency band corresponding to a respiratory process. Additionally or alternatively, the intracranial pulsatile state can include one or more waveform signatures corresponding to the first frequency band or the second frequency band. Furthermore, the abnormal neurological characteristic can be a malfunctioning ventriculoperitoneal (VP) shunt, and/or the abnormal neurological characteristic can be an ineffective endoscopic third ventriculostomy (ETV). Also, the second location can be a finger of the patient, an ear of the patient, or a forehead of the patient.

In some instances, a device to characterize a neurological condition can include a first sensor which can be an optical sensor; and/or a second sensor which can be a biometric sensor. The device can also include a display; at least one processor; and/or at least one memory storing computer-readable instructions that, when executed by the at least one processor, cause the device to perform operations. The operations can include receiving first data representing intracranial waveforms generated by the first sensor at a first location communicatively coupled to a skull of a patient; receiving second data representing a non-cranial biophysical characteristic detected by the second sensor communicatively coupled to a peripheral location on the patient; determining an intracranial pulsatile state of the patient based on the first data and the second data; detecting an abnormal neurological characteristic based on the intracranial pulsatile state; and/or generating an output representing the abnormal neurological characteristic at the display.

In some examples, the second sensor can be a photoplethysmography (PPG), and the non-cranial biophysical characteristic can include a periphery blood flow dynamic represented by a biophysical waveform. Additionally, the device can include a third sensor being an accelerometer. Furthermore, the computer-readable instructions, when executed by the one or more processor, can further cause the device to receive accelerometer data generated by the accelerometer at a third location on the patient; and/or determine a head position using the accelerometer data. The intracranial pulsatile state can be at least partially based on the head position. Also, a third sensor can include an electrocardiogram (ECG), and the computer-readable instructions, when executed by the one or more processor, can further cause the device to generate a cardiac waveform data using the ECG at a third location on the patient; and/or determine a baseline waveform from the cardiac waveform data. Detecting of the abnormal neurological characteristic can be based on comparing the baseline waveform to the first data or the second data. A third sensor, additionally or alternatively, can include an electroencephalography (EEG), and the computer-readable instructions, when executed by the one or more processor, can further cause the device to generate brain activity waveform data using the EEG at a third location on the patient; determine a sleep characteristic from the brain activity waveform data or a wakefulness characteristic from the brain activity waveform data;

and/or determine a seizure characteristic or not seizure characteristic from the brain activity waveform data. The intracranial pulsatile state can be based on the sleep characteristic or wakefulness characteristic affected by the seizure characteristic or not seizure characteristic. Moreover, a third sensor can include a piezo-electric sensor or an inductive sensor, and the computer-readable instructions, when executed by the one or more processor, can further cause the device to generate respiratory waveform data using the piezo-electric sensor or the inductive sensor at a third location on the patient; and/or determine a respiratory pattern from the respiratory waveform data. Detecting of the abnormal neurological characteristic can be based on the respiratory pattern.

In some examples a system to characterize a neurological condition can include a first sensor being a near infrared spectroscopy (NIRS) sensor; a second sensor being a biometric sensor; and/or at least one memory storing computer-readable instructions that, when executed by one or more processor, cause the system to perform operations. The operations can include receiving first data representing intracranial waveforms generated by the first sensor at a first location communicatively coupled to a skull of a patient; receiving second data representing a non-cranial biophysical characteristic detected by the second sensor communicatively coupled to a peripheral neuropathy of the patient; determining an intracranial pulsatile state of the patient based on the first data and the second data; detecting an abnormal neurological characteristic based on the intracranial pulsatile state; and/or causing an output representing the abnormal neurological characteristic to be presented.

In some examples, the computer-readable instructions, when executed by the one or more processor, can further cause the system to identify, using the first data, an indication of a perturbation of the patient in at least one of a first frequency band corresponding to respiratory activity, or a second frequency band corresponding to cardiac activity. Detecting of the abnormal neurological characteristic can be based at least partially on the indication of the perturbation. Also, the system can include an abdominal binder, and the perturbation can include a change of abdominal constriction of the patient using the abdominal binder. Additionally, the perturbation can include a change between a standing position and a laying or siting position; and/or a change between a regular breathing pattern and Valsalva breathing. Furthermore, the system can include a third sensor, the third sensor being an accelerometer, and the computer-readable instructions, when executed by one or more processor, can further cause the system to receive accelerometer data generated by the accelerometer at third location on the patient; and/or determine a head position based on the accelerometer data. The intracranial pulsatile state can include a correlation with the head position. Moreover, detecting of the abnormal neurological characteristic can include determining a first brain pulsation signature based on cerebral hemodynamics represented by the intracranial waveforms correlated with the non-cranial biophysical characteristic; and/or comparing the first brain pulsation signature to a second brain pulsation signature correlated with the non-cranial biophysical characteristic.

The foregoing is intended to be illustrative and is not meant in a limiting sense. Many features of the embodiments may be employed with or without reference to other features of any of the embodiments. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including a hydrocephalus characterization device, which can form at least a part of the system depicted in FIG. 1;

FIG. 2 illustrates an example system including a hydrocephalus characterization device with a patient interface, which can form at least a part of the system depicted in FIG. 1;

FIG. 3 illustrates an example system including a hydrocephalus characterization device with a patient interface having an illumination fiber and a detection fiber, which can form at least a part of the system depicted in FIG. 1;

FIG. 4 illustrates an example system including a hydrocephalus characterization device with a light penetration profile, which can form at least a part of the system depicted in FIG. 1; and

FIG. 5 illustrates an example method for characterizing hydrocephalus, which can be performed by the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the presently disclosed technology may include a variety of combinations and/or integrations of the embodiments described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

Hydrocephalus is a dangerous progressive condition. Missed diagnosis can be life-threatening. CT and MRI scans are currently the best noninvasive diagnostic tests but are often misleading as to whether CSF diversion surgery is needed because static pictures are insufficient to fully understand the complicated dynamic process that is hydrocephalus. There are no accurate, inexpensive, and portable diagnostic tools for hydrocephalus based on intracranial pulsatile dynamics. Some techniques for intracranial characterizations have been based on specialized MRI, invasive ICP monitoring, and CSF infusion/drainage.

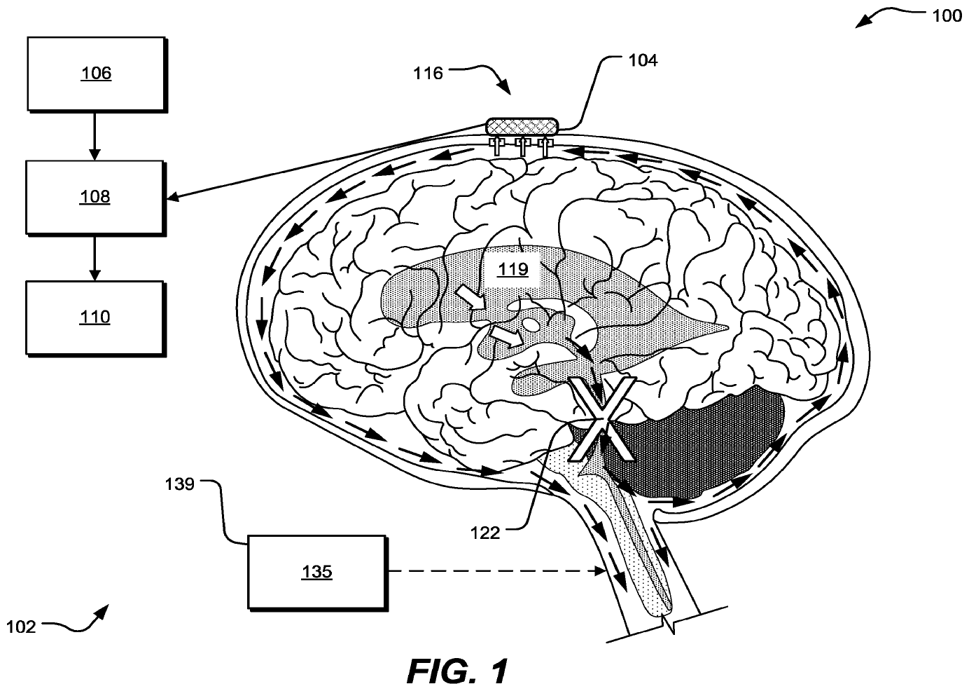

FIG. 1 illustrates an example system 100 including a hydrocephalus characterization device 102 for characterizing hydrocephalus, diagnosing hydrocephalus, and/or detecting an abnormal hydrocephalus characteristic in a patient. The hydrocephalus characterization device 102 can include a first sensor 104 to detect intracranial waveforms generated by intracranial pulsatility of the patient. The first sensor 104 can be a near infrared spectroscopy (NIRS) sensor 105 or other type(s) of optical sensor. The hydrocephalus characterization device 102 can include one or more additional sensors 106, such as a biometric sensor (e.g., a photoplethysmography (PPG) sensor, an accelerometer, an electrocardiogram (ECG), an electroencephalography (EEG), a piezo-electric or inductive respiration sensor, combinations thereof, and the like) to measure additional biometric data. The sensor data can be sent to a hub and/or computing device 108, which can corelate intracranial data from the first sensor 104 with the additional biometric data, and/or perform a spectral analysis on the sensor data to determine an intracranial pulsatile state of the patient. Furthermore, an abnormal hydrocephalus characteristic can be detected based on the intracranial pulsatile state, and an indication of the abnormal hydrocephalus characteristic can be presented at a display 110 of the computing device 108.

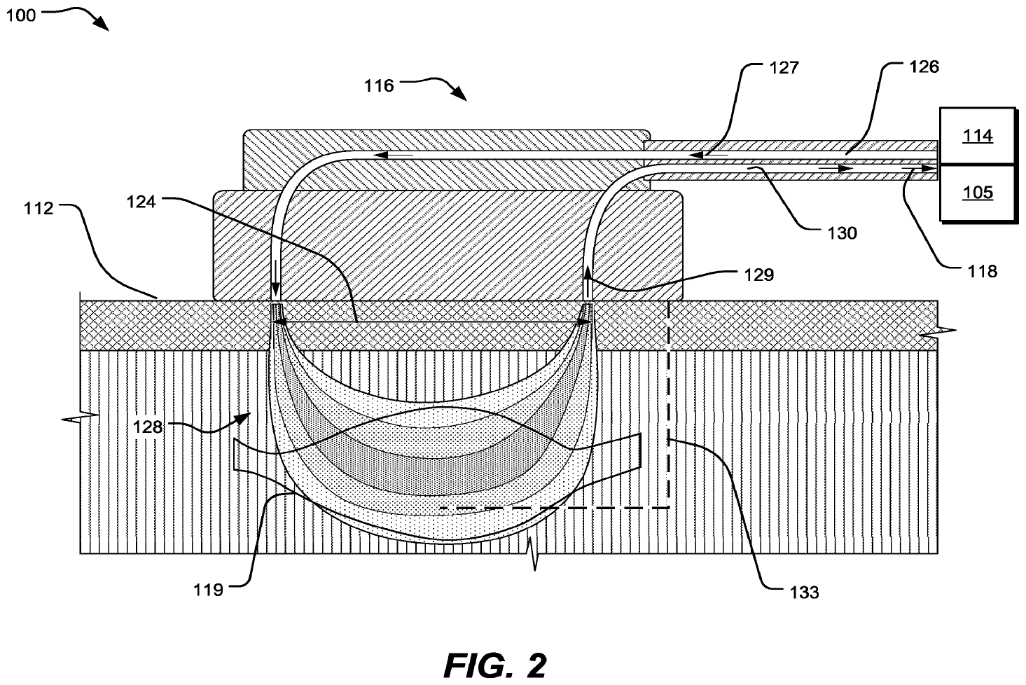

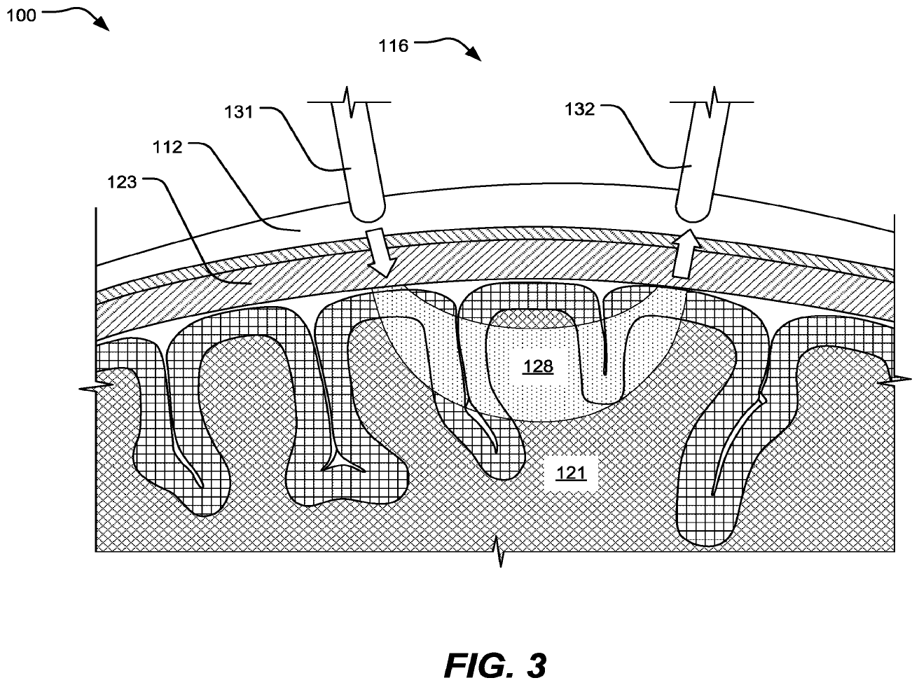

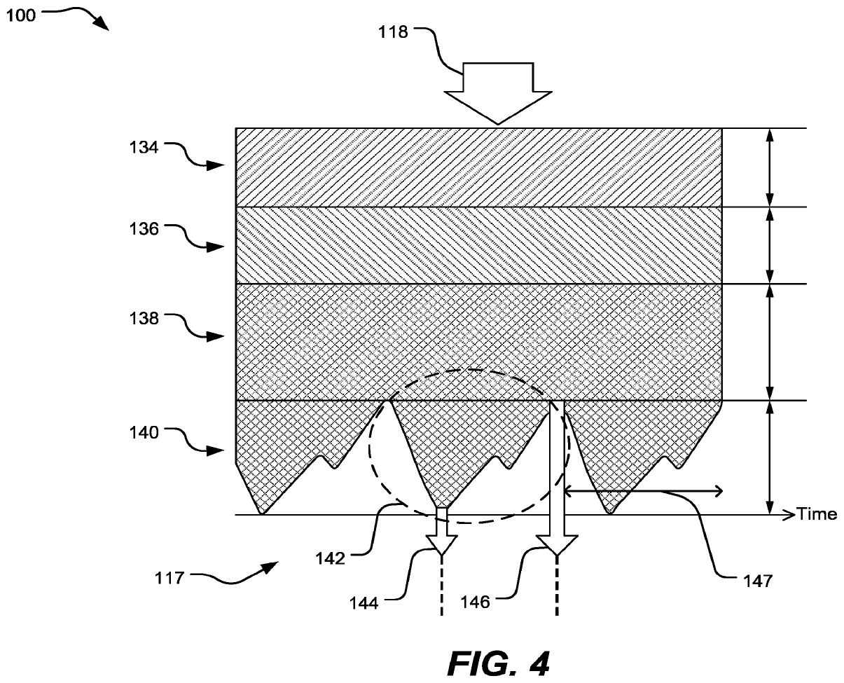

FIGS. 2-4 illustrates examples of the hydrocephalus characterization device 102, portions of the hydrocephalus characterization device 102, and/or various operations performed by the hydrocephalus characterization device 102 to generate the intracranial data. For instance, FIGS. 2 and 3 illustrate example patient interface(s) 116, and FIG. 4 illustrates an example light penetration profile 117. The system(s) 100 depicted in FIGS. 2-4 can be similar to, identical to, and/or can form at least a portion of the system 100 depicted in FIG. 1.

As discussed above, systems, methods, and devices disclosed herein include a diagnostic tool, such as the hydrocephalus characterization device 102 for using noninvasive optical sensors, such as the first sensor 104 (e.g., the NIRS sensor 105), that measure the intensity of light reflected by hemoglobin in the brain and at a peripheral site. The hydrocephalus characterization device 102 can use these techniques to provide better objective evaluation of the status of a patient's hydrocephalus 119 (e.g., by assessing for the presence of shunt malfunction 122 and/or the need for surgical intervention) and can provide for at-home telemetry monitoring to allow earlier detection of shunt malfunction. This can improve patient safety and diagnostic ability. Furthermore, the device can be more broadly applicable to other neurosurgical and neurologic conditions. The device can be a wearable noninvasive diagnostic device for patients with hydrocephalus and/or other neurosurgical and neurologic diseases which uses optical and/or other noninvasive sensors.

In some instances, the hydrocephalus characterization device 102 (e.g., a hydrocephalus diagnostics device) can measure or detect how the brain 121 pulsates within the rigid skull 123 with each heartbeat. Cerebral hemodynamics are unique relative to other organs because the soft brain 121 is encased within the rigid confines of the skull 123, resulting in unique flow impedance properties that can be detected and/or identified with the device 102. It can be determined that changes in CSF and venous pressure within the rigid skull 123 have a larger effect on intracranial than peripheral pulsatile flow dynamics in the cardiac and respiratory frequency bands. Hydrocephalus disrupts the healthy pulsatile equilibrium between the volume of the brain 121, blood, and cerebrospinal fluid (CSF). This principle can serve as the basis for diagnostic determinations using the device.

Other techniques that focus on the effects of hydrocephalus and CSF drainage on brain oxygenation levels over longer time scales (by comparing average brain hemoglobin and deoxyhemoglobin concentrations) can be less effective as a diagnostic approach than those measuring cerebral hemodynamics. Some of the techniques described herein focus on the dynamic relationships between these pulse waveforms in the cardiac and respiratory frequency bands to characterize an “intracranial pulsatile state.”

For instance, normal pulsatile dynamics depend on a healthy pulsatile equilibrium between the volume of brain, blood, and cerebrospinal fluid (CSF), and are influenced by the respiratory and cardiac cycles. Anatomic or physiologic disturbance of this equilibrium can lead to progressive (and dangerous) conditions, some of which can be treated with neurosurgical procedures to restore more healthy pulsatile dynamics. The systems, methods, and devices disclosed herein use near infrared spectroscopy (NIRS) techniques and/or similar optical-based techniques to better monitor, understand, and guide treatment of neurosurgical conditions at a diagnostic level. NIRS is a safe, inexpensive, noninvasive, and portable technique for studying pulsatile cerebrovascular dynamics and/or determining the intracranial pulsatile state.

In some instances, the system, method, and/or device uses optical and other noninvasive physiologic sensors to differentiate controlled versus uncontrolled hydrocephalus (and other neurosurgical and neurologic disease states) by characterizing intracranial pulsatility (ICP). ICP can be one of multiple variables affecting the pulsatile state. Additional information may be determined and incorporated into the intracranial pulsatility, such as by distinguishing between a low pressure or a high-pressure type of hydrocephalus.

CSF diversion procedures for hydrocephalus cost approximately $2 billion annually in the United States and are projected to grow to approximately $4 billion by 2030. Patients with hydrocephalus undergo regular checkups with routine imaging (CT or MRI), and are frequently evaluated in the emergency department with additional imaging when they have symptoms concerning for shunt malfunction. CT and MRI are the primary noninvasive tests used in workup. However, these scans are costly (>$1000), and as discussed above, are not particularly sensitive for shunt malfunction. An inexpensive, noninvasive, wearable device that detects progressive hydrocephalus (e.g., shunt malfunction) is in heavy demand because it can improve patient safety, reduce the cost of care, improve diagnostic accuracy, and improve quality of life for patients.

There are many unanswered clinical neurosurgical questions, and neurosurgical treatment decisions often depend on the surgeon's judgment, experience, and clinical philosophy in the face of limited objective data. For example, determining whether a ventriculoperitoneal (VP) shunt is malfunctioning can be a diagnostic dilemma. MRI techniques may not be able to reliably identify which Chiari patients will be symptomatic and why, and it is unknown why some cases of “communicating hydrocephalus” are effectively treated by an endoscopic third ventriculostomy (ETV) and others are not.

In some scenarios, the techniques disclosed herein can be used to diagnose and/or treat patients with hydrocephalus treated with ventriculoperitoneal (VP) shunt; hydrocephalus treated with endoscopic third ventriculostomy (ETV) (e.g., an additional pathway for pulsatile CSF flow is created at the floor of the third ventricle); patients with Chiari Type I malformation; neurosurgical patients with an intracranial pressure (ICP) monitor or an external ventricular drain (EVD); combinations thereof, or the like.

Hydrocephalus diagnostics can be improved by using near infrared techniques because they are more portable, less expensive, less cumbersome, and easier to operate than MRI, transcranial doppler (TCD), and invasive ICP monitoring, and can provide detailed information about intracranial pulsatility. Cerebral hemodynamic variation can be detected and characterized by comparing near infrared waveforms with continuous blood pressure recordings using spectral analysis techniques such as the continuous wavelet transform and wavelet coherence. Spectral analysis techniques can be used in these analyses, though the shape of the beat-to-beat waveforms can also carry information about the intracranial pulsatile state.

In some instances, the systems, methods, and devices disclosed herein can be used to classify cerebral hemodynamic signals into six frequency bands that represent various physiologic processes that occur over different timescales.

For instance, a first band (Band I) in the 0.4-2 Hz range can correspond to cardiac processes. A second band (Band II) in the 0.15-0.4 Hz range can correspond to respiratory processes. A third band (Band III) in the 0.05-0.15 Hz range can correspond to smooth muscle activity in resistance vessels and may be partially under autonomic control. A fourth band (Band IV) in the 0.02-0.05 Hz range can correspond to smooth muscle activity, and/or tight neurovascular coupling under autonomic control. A fifth band (Band V) in the 0.0095-0.02 Hz range can correspond to nitric oxide related endothelial activity or endothelial metabolic activity. A sixth band (Band VI) in the 0.005-0.0095 Hz range can correspond to nitric oxide independent of endothelial activity.

The systems, methods, and devices can determine that both the cardiac and respiratory cycles affect CSF pulsatility, and with the most variation related to respiration. Respiration can affect intrathoracic pressure, cerebral venous return, and the equilibrium between the pressure in the spinal epidural venous plexus and the thecal sac, thus affecting CSF flow across the foramen magnum and cerebral venous drainage. In addition, impairment of cerebral venous outflow can be determined to result in increased pulsatile flow through the cerebral aqueduct. Perturbations of intracranial pulsatility through the variation of normal physiologic processes (breathing, posture, end tidal CO2, Valsalva) can be detected and characterized with NIRS based on these determinations.

In some examples, NIRS and peripheral photoplethysmography can be used to detect perturbations of the intracranial mechanical resonant state caused by neurosurgical conditions and treatments. Though NIRS and peripheral photoplethysmography waveforms in frequency bands I and II are highly coherent, it can be determined that neurosurgical pathology can also have detectable signatures in these bands, and that waveform morphology can carry additional useful information, which can lead to a better mechanistic understanding that improves diagnosis and treatment.

Intracranial pulsatility can be determined by comparing and/or correlating cerebral NIRS waveforms with peripheral (finger, ear lobe, forehead, or other location) photoplethysmography (PPG) waveforms, with a focus on the cardiac and respiratory frequency bands. These sensors are noninvasive, low power, portable, and use red and near infrared wavelengths to measure oxyhemoglobin and deoxyhemoglobin absorbance with high time resolution. The intracranial pulsatility can be further characterized and/or correlated to peripheral PPG waveforms using normal physiologic perturbation of CSF and venous systems by altering posture (laying, squatting, standing), respiration (Valsalva, breathing rate), and/or abdominal binder (elastic band applied around the abdomen). In some examples, an integrated device (e.g., with NIRS, PPG, accelerometer, EEG) can measure the different data types from the forehead rather than from multiple body sites. For instance, the peripheral PPG waveform can be measured from the forehead right next to the NIRS sensor (e.g., with the difference in what is measured resulting from the sensor geometry)

In some examples, the techniques disclosed herein generate finger and/or earlobe photoplethysmography, as well as forehead NIRS recordings, before and after neurosurgical intervention, and at long term follow up at least 3-6 months after surgery. The inclusion of patients with an ICP monitor or EVD can provide a correlation of the ICP waveform with the NIRS and peripheral sensor waveforms.

In some instances, an example hydrocephalus characterization device 102 includes (1) a Cerebral NIRS 105 which can be held in place on the forehead or other portion of epidermis 112 on the patient's head with an elastic band; (2) a peripheral PPG (e.g., the one or more additional sensors 106) which can include a finger clip, ear clip, or other site (e.g., for comparison of multiple peripheral sites); (3) an accelerometer (ACC) to continuously record head position; (4) an electrocardiogram (ECG) to provide a common reference point for a start of a cardiac cycle for waveform analysis; (5) an Electroencephalography (EEG) to provide information on brain electrical activity and sleep/wake status; (6) a Piezo-electric or inductive respiration sensor for measuring respiratory activity; (6) an event trigger switch to create a binary output to mark specific times of events of interest; and/or (7) an intracranial pressure (ICP) monitor adaptor.

Furthermore, the hydrocephalus characterization device 102 can include a lightweight, battery-powered, hub (e.g., formed of plastic) with multiple plug and play sensor ports into which the individual sensors connect. The hub can include the computing device 108 which receives the data generated by the sensors as inputs, and/or can convert the inputs into outputs, which the hub communicates (e.g., wirelessly using Bluetooth) to data collection software (e.g., executing on laptop or desktop computer). In some instances, the hydrocephalus characterization device 102 uses a sampling frequency of 300 Hz.

The techniques disclosed herein can arrange the sensors in an optimal configuration for measuring various biomechanical traits from which the intracranial pulsatile state of the patient is determined. The primary sensors used by the hydrocephalus characterization device 102 can be the NIRS sensor 105, PPG, and accelerometer. The EEG, ECG, and respiration sensor(s) can be used as secondary or supplementary sensors (e.g., the one or more additional sensors 106) for further improvement or optimization on generating the intracranial pulsatile state profile. The sensors of the hydrocephalus characterization device 102 and their operations are discussed in greater detail below.

Turning to FIGS. 2 and 3, in some scenarios, the hydrocephalus characterization device 102 includes one or more Near-Infrared Spectroscopy (NIRS) sensor(s) 105. A NIRS sensor 105 can include a light source 114 and/or a photodetector 115 which can be separated by a predetermined distance 124 (e.g., between 10-30 mm, such as 25 mm). The NIRS sensor 105 can be placed on the scalp. The NIRS sensor 105 can be coupled to a patient interface 116 and can shine light through the scalp and skull 123. For instance, the hydrocephalus characterization device 102 can include a first light channel 126 coupled to the light source 114 for providing incident light 127 into the detection area 128. The NIRS sensor 105 can detect light 118 reflected off of the oxyhemoglobin and deoxyhemoglobin flowing through the brain 121 to generate a pulsatile waveform, for instance, by receiving an output light 129 through a second light channel 130. In some examples, the first light channel 126 and/or the second light channel 130 can include one or more optical fibers. For instance, the first light channel 126 can be formed by a first optical fiber, or an illumination fiber 131. The second light channel 130 can be formed of a second optical fiber, or a detection fiber 132. Furthermore, the hydrocephalus characterization device 102 can include the peripheral PPG (e.g., as the one or more additional sensors 106). The peripheral PPG can include the light source 114 and photodetector arranged so that light reflected off of oxyhemoglobin and deoxyhemoglobin flowing under the sensor is detected. By way of example, the light 118 can have a detection penetration depth 133 corresponding to a 95% signal threshold. The penetration depth can be between 10-30 mm, such as 23 mm. Furthermore, the system 100 can include a perturbation 135, such as an abdominal binder 139 changing an abdominal constriction, a change between a standing position and a laying or sitting position, or a change between a regular breathing pattern and Valsalva breathing

In some instances, the hydrocephalus characterization device 102 can include the accelerometer (e.g., as the one or more additional sensors 106), which can be secured to the head of the patient so that head position and physical activity can be tracked. Additionally, the one or more additional sensors 106 of the hydrocephalus characterization device 102 can include the EEG, which can be a limited channel scalp EEG and can be incorporated into the sensor contact surface. Moreover, the one or more additional sensors 106 of the hydrocephalus characterization device 102 can include the ECG, which can be a limited channel ECG and can be incorporated into the sensor contact surface or a device on another body part that communicates with the primary device via wire or telemetry.

In some examples, the NIRS sensor 105 can include a light emitting diode as the light source 114 and/or a photodiode sensor housed in soft rubber. The NIRS sensor 105 can be gently held in place over the forehead with an elastic headband. The periphery photoplethysmography (PPG) sensor can be held in a finger clip or can be clipped to the ear lobe. The accelerometer can be attached to the elastic headband to continuously record head position. The room lights can be turned down to minimize contamination from ambient light. After sensor positioning, the signal acquisition can be initiated, and the data can be recorded to the computing device 108 (e.g., a desktop or laptop computer) during the acquisition.

Turning to FIG. 4, in some examples, the light 118 can penetrate different anatomical layers of the patient at different depths to form the light penetration profile 117. The light penetration profile 117 of FIG. 4 depicts an amount of penetration of the light 118 through the anatomical layers over time.

For instance, the light 118 can penetrate a first anatomical layer such as tissue layer 134, for instance, with full or nearly full penetration. As such, the tissue layer 134 can have a consistent light penetration over time. Next, the light 118 can penetrate a second anatomical layer such as a venous blood layer 136. The venous blood layer 136 can also be fully or nearly fully penetrated with the light 118 and/or can have consistent light penetration over time. Below the venous blood layer 136, the light 118 can penetrate a third anatomical layer such as a non-pulsatile artery blood layer 138. The non-pulsatile artery blood layer 138 can be fully or nearly fully penetrated by the light 118 and/or can have consistent light penetration over time. Furthermore, the anatomical layers can include a fourth anatomical layer such as a pulsatile blood layer 140 (e.g., below the non-pulsatile artery blood layer 138). The pulsatile blood layer 140 can have full penetration or partial penetration from the light 118. Moreover, the light penetration at the pulsatile blood layer 140 can fluctuate periodically over time, with a rhythmic pattern, such as a beat-to-beat waveform signature 142, detectable between a systolic phase 144 and a diastolic phase 146. The beat-to-beat waveform signature 142 can have one or more frequency bands 147, such as first frequency band corresponding to a cardiac process; a second frequency corresponding to a respiratory process, and/or another frequency band, as discussed above.

In some examples, the data collection procedure can include recording data for 5-10 minutes, during which time the subject can lay supine, stand upright, breath at different rates, and don and doff an elastic abdominal binder at 30 second to 1-minute intervals (e.g., the perturbation 135). to allow the patient to reach physiologic equilibrium. The sensors can be removed, and the data file can be saved for later analysis. The equipment can be cleaned with antiseptic wipes after each use. The laptop hard drive used to record data from the sensors can be encrypted and identifying patient information can be omitted from storage on the laptop.

In some instances, the NIRS and PPG sensors use two wavelengths in the red and near-infrared bands, optimized for oxyhemoglobin and deoxyhemoglobin, but can be designed for other specific chromophores such as cytochrome oxidase C. The NIRS waveform can represent a direct measure of hemoglobin in the blood, rather than CSF pulsatility. However, the measure of hemoglobin can be correlated to the CSF pulsatility. Other photosensor techniques based on similar principles may be used in the hydrocephalus diagnostics device. For example, near infrared transillumination backscattering sounding to measure subarachnoid space width oscillations is a variation of the NIRS sensor geometry, wavelengths, and technical aspects of signal acquisition. Additional or alternative optical sensor techniques that can be used include continuous wave NIRS, frequency domain NIRS, time domain NIRS, broadband NIRS, diffuse correlation spectroscopy, combinations thereof, and the like.

The cerebral NIRS sensors 105 used for clinical applications can sample on the order of 1-2 Hz, and trends over seconds, minutes, and hours can be interpreted clinically. In some instances, the techniques disclosed herein use at least 50 Hz sampling to generate a pulsatility waveform so that the beat-to-beat waveforms and cardiac and respiratory frequency bands can be analyzed.

In some instances, EEG can be used to determine sleep versus wake states. This can contribute to the hydrocephalus diagnostics because autonomic regulation of cerebral blood flow during sleep-wake cycles can affect brain volume, global intracranial pressure, and intracranial pulsatile dynamics. For instance, he EEG sensor that can be implemented to detect a sleep and/or wake state as well as major seizures. Sometimes patients with shunt malfunction display whole body muscle “posturing” activity that is confused for seizures. The EEG component of the device may differentiate posturing due to high intracranial pressure caused by shunt malfunction from seizure activity. Furthermore, the ECG can be used as a reference point for phase-related comparisons of NIRS and PPG to increase accuracy of waveform comparisons. ECG waveform morphology can be compared to NIRS and PPG waveforms.

In some scenarios, the device components, including the various sensors discussed herein, can be integrated in a one-piece housing that attaches to the patient's head to form the interface(s) 116, or can be configured as several discrete modules placed in several locations on the patient's body that communicate wirelessly and/or using wired connections. The modules can be powered by a battery or wirelessly. The physiologic data can be displayed to the patient on a local device and/or transmitted via a network connection to the treating physician for evaluation.

As discussed above, the NIRS sensor 105 can be placed on the forehead, and the PPG sensor can be placed on the ear lobe, forehead, finger, or wrist. Other configurations can be arranged to add additional information. For instance, the NIRS sensor 105 may be placed on other parts of the head, or multiple NIRS sensors 105 can be used to monitor different locations on the head. The PPG sensor can be positioned to record peripheral blood flow dynamics on nearly any part of the body.

In some examples, the hydrocephalus characterization device 102 can incorporate an accelerometer sensor to continuously monitor head position and/or activity (walking, resting, head rotation). The device-skin contact surface can also incorporate limited-channel surface EEG to allow detection of a sleep stage and other brain activity, and as described above ECG can be incorporated.

In some instances, the hydrocephalus characterization device 102 can be light weight, low power, low profile, and/or wearable to allow continuous, long term data collection. The batteries can be replaced or recharged. Furthermore, the hydrocephalus characterization device 102 can have internal memory to record data. Data downloading or streaming, and device configuration can, in some instances, be performed wirelessly using Bluetooth or another wireless interface, or through a wired connection port. The device can interface with software running on a smartphone, tablet, local computer, or cloud platform for configuration, data viewing, and remote interpretation.

Data analysis performed by the systems, methods, and devices disclosed herein can focus on NIRS and PPG data sampled at least 50 Hz to generate pulse waveforms that can be analyzed in the cardiac-respiratory frequency bands. In some instances, a frequency of less than 50 Hz (e.g., between 10 Hz and 50 Hz) can be used as a sampling frequency, for instance, to conserve bandwidth. Primary time scales of interest can be shorter than the time scales at which biochemical and neuroregulatory mechanisms affect cerebral blood flow, thus resulting in the detection of the dynamic instantaneous mechanical relationship between volume, movement, and pressure of the brain 121, CSF, and blood.

In some examples, the NIRS and PPG waveforms are compared using spectral techniques (e.g., wavelet analyses), and direct beat-to-beat phase, amplitude, and waveform shape analyses. Additional information is gained through a “perturbation protocol” in which the system is noninvasively perturbed with changes in posture and venous pressure using an elastic abdominal binder. These transient perturbations can cause shifts in CSF and venous blood, which can affect intracranial flow impedance and therefore pulsatility. Wearing the hydrocephalus characterization device 102 during various activities of daily living can also capture perturbations of the system which provide for more thorough characterization of the “intracranial pulsatile state.” The accelerometer and EEG components of the device can detect these activities and head positions, which can be correlated to the perturbations detected by the NIRS and PPG sensors. From this data, parameters that describe the “intracranial pulsatile state” can be defined, and healthy and pathologic states can be classified. For example, shunt malfunction versus healthy and safe intracranial pulsatile dynamics, or problematic versus asymptomatic Chiari malformation can be detected and identified.

Though the intracranial pulsatile state can be based on dynamics at the cardiac and respiratory frequencies, the data collected by the device at the >50 Hz sampling frequency can also contain the information necessary to analyze lower frequency trends in NIRS relative to PPG that relate to biochemical and autonomic regulation of cerebral blood flow, which also affect intracranial pulsatility.

In some examples, continuous monitoring in and out of the hospital may be possible with the hydrocephalus characterization device 102 compared to some imaging techniques that use a large machine to produce a snapshot at a single point in time. Collecting data from different times of the day and physical activities can give a more comprehensive picture of the intracranial state and can provide for earlier detection of a potential problem that merits early workup. The sensors discussed herein can be relatively inexpensive, further lowering the barriers to implementing the hydrocephalus characterization device 102.

In some instances, a patient can alternate between a supine position and standing while data is collected with the NIRS, PPG, and accelerometer. Accelerometer changes correlated with changes in the magnitude of the 1-dimensional continuous wavelet transform and wavelet cross spectrum analysis can be detected. Furthermore, the patient may alternate between Valsalva and regular breathing, which can create measurable data points or signatures in the NRS and/or PPG data. Differences in spectral analyses (1D wavelet, cross-wavelet transforms magnitude and phase) can be detected in cardiac and respiratory frequency bands responsive to the alternating breathing.

Furthermore, data can be collected and analyzed corresponding to an upright position with constant breathing rate while taking the abdominal binder on and off. For instance, a magnitude of continuous wavelet transform can be different with an abdominal binder on versus off for NIRS waveform but not for PPG waveform, supporting the idea that perturbation in systemic venous pressure affects intracranial more than peripheral pulsatile dynamics. Wavelet coherence analysis of NIRS vs PPG can show different patterns of magnitude squared coherence and phase with an abdominal binder on versus off. Coherence analysis comparing NIRS-red and NIRS-infrared waveforms can show differences in magnitude and phase with binder on and off.

As noted above, other neurosurgical and neurologic diseases can be detected, measured, analyzed, and/or diagnosed, such as Chiari malformation, pseudotumor cerebri, and multiple sclerosis, which have disturbed pulsatile dynamics, as well as neurovascular dysregulation, which occurs in migraine headaches.

In some examples, data and statistical analysis can be performed with MATLAB mathematical analysis software (e.g., MathWorks, Natick, MA) and/or Python numerical analysis packages. After appropriate preprocessing (truncation, detrending, filtering), spectral analysis of amplitude vs time waveforms can be performed (Fourier transform, continuous wavelet transform, wavelet coherence analysis of cross wavelet power and phase), as well as analysis of variation of beat-to-beat waveform morphology, phase, and amplitude. The analyses can compare pre- and post-treatment measurements and well as subjects without neurosurgical conditions. The Wilcoxon rank sum test can be used to compare continuous variables. When applicable, a p-value of less than 0.05 can be considered statistically significant.

As discussed above, the gelatinous brain 121 pulsates within the rigid skull 123 with each heartbeat, resulting in unique cerebral hemodynamics relative to other organs. Hydrocephalus disrupts the healthy pulsatile equilibrium between the volume of the brain, blood, and cerebrospinal fluid (CSF). Using the techniques disclosed herein, changes in this equilibrium can be noninvasively detected by comparing intracranial and peripheral pulsatile dynamics in the cardiorespiratory frequency band. Using cerebral near infrared spectroscopy and peripheral photoplethysmography sensors to detect light reflected by hemoglobin, the device can collect various types of physiological/biomechanical data. NIRS and PPG waveforms can be compared with spectral techniques (wavelet) and direct beat-to-beat waveform analysis to define an individual's “intracranial pulsatile state,” comprising of cardiorespiratory frequency differences in magnitude, phase, and waveform shape in upright and supine postures, and at normal and mildly increased central venous pressure (using an elastic abdominal binder). A classification model based on these parameters can be created to classify the intracranial pulsatile state as healthy or pathologic.

In other words, sensors placed on the forehead and finger or earlobe continuously shine light and measure how much light is reflected by hemoglobin in the blood. Pilot data can be collected and/or associated with each of the following groups: children with hydrocephalus before and after surgery, children without hydrocephalus, and inpatients with invasive intracranial pressure (ICP) monitors placed as part of routine treatment.

Data can be studied with the techniques disclosed herein to detect beat-to-beat patterns. Measurements taken at different postures and venous blood pressures (by wearing an elastic abdominal binder) can give additional information to help understand healthy versus not-healthy patterns.

The techniques disclosed herein can be used to assess intracranial pulsatility using optical near infrared spectroscopy sensors that continuously monitor light reflected by hemoglobin in the blood flowing through the brain 121 and peripheral vessels. Pulsatile blood flow can be correlated with CSF pulsatility due to the conservation of the total volume of blood, brain 121, and CSF within the skull 123.

The techniques can be used to study mechanical properties of intracranial pulsatility in the higher frequency cardiorespiratory band. An “intracranial pulsatile state” can be defined for a specific patient at a specific time as the set of absolute and relative magnitude, phase, and waveform shape parameters derived from brain 121 and peripheral sensor data. Models, such as circular-linear regression and multidimensional support vector machines, can be compared to classify the intracranial pulsatile state as normal or pathologic (e.g., needing CSF diversion surgery). Both peripheral and intracranial hemodynamics can be deployed to classify CSF physiology as pathologic or healthy.

One or more methods disclosed herein can involve building a model to describe normal intracranial pulsatile states in healthy people by comparing brain and peripheral blood pulsations using transcutaneous optical sensors. For instance, cerebral near-infrared spectroscopy on the forehead and peripheral photoplethysmography waveforms on the finger and earlobe can be recorded. Posture (e.g., standing, supine) and central venous pressure (e.g., elastic abdominal binder on and off) can be varied. The intracranial-extracranial difference in magnitude, phase, and waveform shape at cardiac frequencies can be calculated using spectral (wavelet) and direct waveform feature analyses to define the intracranial pulsatile state. The variability across patients can be assessed. Normal time ranges for the transition between states can be calculated. Waveforms from invasive ICP monitors (e.g., implanted as part of routine care) can be compared to noninvasive sensors using these techniques. As such, it can be determined whether intracranial pulsatile state is maintained within a normal range across healthy subjects.

In some instances, the one or more methods can classify intracranial pulsatile state in patients with hydrocephalus as distinctly healthy or pathologic. For instance, the intracranial pulsatile state before and after hydrocephalus surgery can be monitored to track the response to intervention. Then the ability of classification models such as linear-circular regression and multidimensional support vector machines to classify the pre-and post-operative states as pathologic or healthy can be compared for accuracy and/or analytical optimization. As such, it can be determined that effectively treated hydrocephalus, symptomatic hydrocephalus, and normal controls have unique pulsatile states.

In some examples, the one or more methods can include placing the NIRS sensor on the forehead of the patient, the PPG sensor on finger or ear or forehead, the accelerometer connected to NIRS sensor on forehead, and/or the ECG stickers and respiration sensor on the chest. Physiologic data can be recorded for 2 minutes in a supine position, then 2 minutes in a standing position (e.g., with no abdominal binder). The abdominal binder can be applied. Then the physiological data can be recorded for 2 minutes in the supine position, then for 2 minutes in the standing position. The abdominal binder can be removed while standing. Physiological data can be recorded for 2 more minutes. As such, the total recording time for this data collection session can be between 10-15 minutes.

In some examples, the two-minute segments can allow hemodynamics from posture and abdominal binder changes to reach steady state. Data can be saved for later analysis. Perturbation of the pulsatile system with posture changes and venous pressure changes (abdominal binder) can elicit more information about system pulsatility characteristics (steady state and state transition) then without these perturbations.

In some examples, data can be analyzed with MATLAB mathematical analysis software (MathWorks, Natick, MA). Statistical analysis can be performed with MATLAB and R. Raw data can be converted to meaningful units based on transfer functions in each sensor's specification manual. The resultant waveforms can have a characteristic shape that repeats every cardiac cycle and varies in morphology, amplitude, and phase depending on physiologic conditions. NIRS, PPG, and ICP waveforms can be analyzed using several techniques, discussed below.

Wavelet analyses with specific focus on the cardiorespiratory frequency range (0.1 Hz to 3 Hz) can be performed. This can include (a) the continuous wavelet transform being applied to individual NIRS, PPG, and ICP signals to study the time variation in the frequency domain of magnitude and phase of component frequencies; (b) the cross-wavelet transform being applied to NIRS, PPG, and ICP waveforms to compare magnitudes and phases in the frequency domain over time; and/or (c) differences between NIRS and PPG magnitude and phase over time being calculated.

Direct analysis of beat-to-beat phase, amplitude, and waveform shape can be performed. This technique can provide direct calculation of instantaneous heart rate, phase, and amplitude and can be useful for state transition analysis (see below).

Steady state analysis can be performed. For instance, differences between magnitudes and phases of signal components in the cardiorespiratory frequency band can be compared across steady states (posture, +/−abdominal binder). This can result in a set of magnitudes and phases corresponding to a time segment during data acquisition whose means and variation can be compared. The Shapiro test can be used to calculate normality of the distribution of the data. Additionally or alternatively, a Mann-Whitney U test can be used to compare means of nonparametric data.

State transition analysis can be performed, such that a time to reach steady state, fluctuation in magnitude and phase of cardiorespiratory frequency components (accounting for variation in heart rate) after changing state (posture or abdominal binder) until new equilibrium is reached, and/or rate of change of magnitude and phase of signals can be determined. Furthermore, data can be tagged with push-button event data, and continuous accelerometer data can track posture. This can help with automation of data analysis.

Intracranial pulsatile state (IPS) analysis can be performed, which can include an analysis of each measurement session using the above techniques and can result in a set of NIRS and PPG magnitudes, phases, and waveform shape (absolute and relative values) associated with varying postures, central venous pressures (abdominal binder on/off), and underlying clinical condition (pre/post-op, +/−hydrocephalus). This set of parameters for a specific patient under specific conditions can be defined as the intracranial pulsatile state.

Furthermore, classification models, such as linear-circular regression and support vector machines, can be compared in their ability to accurately classify the intracranial pulsatile state as pathologic, healthy, working shunt present, and/or no shunt present.

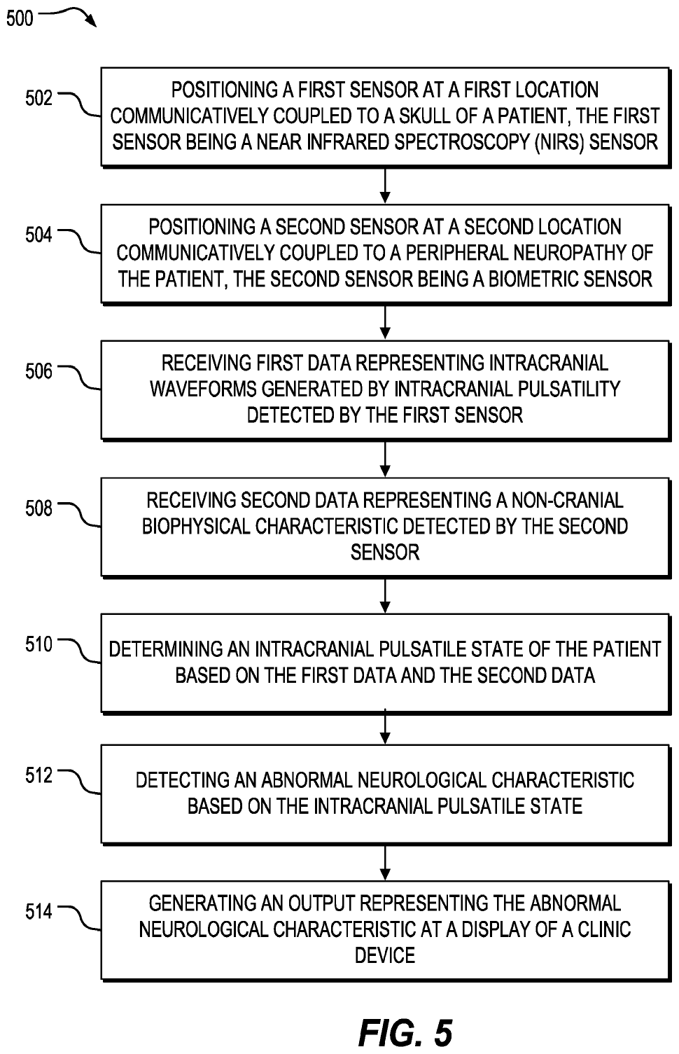

Turning to FIG. 5, an example method 500 for characterizing a neurological condition is depicted. The method 500 can be performed by and/or can form a portion of any of the system(s) 100 disclosed above regarding FIGS. 1-4.

In some examples, at operation 502, the method 500 can position a first sensor at a first location communicatively coupled to a skull of a patient, the first sensor being a near infrared spectroscopy (NIRS) sensor. At operation 504, the method 500 can position a second sensor at a second location communicatively coupled to a peripheral neuropathy of the patient, the second sensor being a biometric sensor. At operation 506, the method 500 can receive first data representing intracranial waveforms generated by intracranial pulsatility detected by the first sensor. At operation 508, the method 500 can receive second data representing a non-cranial biophysical characteristic detected by the second sensor. At operation 510, the method 500 can determine an intracranial pulsatile state of the patient based on the first data and the second data. At operation 512, the method 500 can detect an abnormal neurological characteristic based on the intracranial pulsatile state. At operation 514, the method 500 can generate an output representing the abnormal neurological characteristic at a display of a clinic device.

It is to be understood that the specific order or hierarchy of steps in the method(s) depicted in FIG. 5 and throughout this disclosure are instances of example approaches and can be rearranged while remaining within the disclosed subject matter. For instance, any of the operations depicted in FIG. 5 and throughout this disclosure may be omitted, repeated, performed in parallel, performed in a different order, and/or combined with any other of the operations depicted in FIG. 5 and throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method to characterize a neurological condition, the method comprising:
positioning a first sensor at a first location communicatively coupled to a skull of a patient, the first sensor being a near infrared spectroscopy (NIRS) sensor;
positioning a second sensor at a second location communicatively coupled to a peripheral neuropathy of the patient, the second sensor being a biometric sensor;
receiving first data representing intracranial waveforms generated by intracranial pulsatility detected by the first sensor;
receiving second data representing a non-cranial biophysical characteristic detected by the second sensor;
determining an intracranial pulsatile state of the patient based on the first data and the second data;
detecting an abnormal neurological characteristic based on the intracranial pulsatile state; and
generating an output representing the abnormal neurological characteristic at a display of a clinic device.

2. The method of claim 1, further comprising:
performing a spectral analysis on the first data to determine one or more beat-to-beat waveform signatures representing intracranial activity,
wherein,
the one or more beat-to-beat waveform signatures are correlated to the non-cranial biophysical characteristic to detect the abnormal neurological characteristic.

3. The method of claim 1,
wherein,
detecting of the abnormal neurological characteristic includes comparing the intracranial pulsatile state of the patient to a reference intracranial pulsatile state based on previously generated data.

4. The method of claim 1, further comprising:
determining a first frequency band corresponding to a cardiac process; and
determining a second frequency band corresponding to a respiratory process,
wherein,
the intracranial pulsatile state includes one or more waveform signatures corresponding to the first frequency band or the second frequency band.

5. The method of claim 1,
wherein,
the abnormal neurological characteristic is a malfunctioning ventriculoperitoneal (VP) shunt.

6. The method of claim 1,
wherein,
the abnormal neurological characteristic is an ineffective endoscopic third ventriculostomy (ETV).

7. The method of claim 1,
wherein,
the second location is a finger of the patient, an ear of the patient, or a forehead of the patient.

8. A device to characterize a neurological condition, the device comprising:
a first sensor, the first sensor being an optical sensor;
a second sensor, the second sensor being a biometric sensor;
a display;
at least one processor; and
at least one memory storing computer-readable instructions that, when executed by the at least one processor, cause the device to:
receive first data representing intracranial waveforms generated by the first sensor at a first location communicatively coupled to a skull of a patient;
receive second data representing a non-cranial biophysical characteristic detected by the second sensor communicatively coupled to a peripheral location on the patient;
determine an intracranial pulsatile state of the patient based on the first data and the second data;
detect an abnormal neurological characteristic based on the intracranial pulsatile state; and
generate an output representing the abnormal neurological characteristic at the display.

9. The device of claim 8,
wherein,
the second sensor is a photoplethysmography (PPG), and
the non-cranial biophysical characteristic includes a periphery blood flow dynamic represented by a biophysical waveform.

10. The device of claim 8, further comprising:
a third sensor, the third sensor being an accelerometer,
wherein,
the computer-readable instructions, when executed by the one or more processor, further cause the device to:
receive accelerometer data generated by the accelerometer at a third location on the patient; and
determine a head position using the accelerometer data, the intracranial pulsatile state is at least partially based on the head position.

11. The device of claim 8, further comprising:
a third sensor including an electrocardiogram (ECG),
wherein,
the computer-readable instructions, when executed by the one or more processor, further cause the device to:
generate a cardiac waveform data using the ECG at a third location on the patient; and
determine a baseline waveform from the cardiac waveform data, detecting of the abnormal neurological characteristic is based on comparing the baseline waveform to the first data or the second data.

12. The device of claim 8, further comprising:
a third sensor including an electroencephalography (EEG),
wherein,
the computer-readable instructions, when executed by the one or more processor, further cause the device to:
generate brain activity waveform data using the EEG at a third location on the patient;
determine a sleep characteristic from the brain activity waveform data or a wakefulness characteristic from the brain activity waveform data; and
determine a seizure characteristic or not seizure characteristic from the brain activity waveform data, the intracranial pulsatile state is based on the sleep characteristic or wakefulness characteristic affected by the seizure characteristic or not seizure characteristic.

13. The device of claim 8, further comprising:
a third sensor including a piezo-electric sensor or an inductive sensor,
wherein,
the computer-readable instructions, when executed by the one or more processor, further cause the device to:
generate respiratory waveform data using the piezo-electric sensor or the inductive sensor at a third location on the patient; and
determine a respiratory pattern from the respiratory waveform data, detecting of the abnormal neurological characteristic is based on the respiratory pattern.

14. A system to characterize a neurological condition, the system comprising;
a first sensor, the first sensor being a near infrared spectroscopy (NIRS) sensor;
a second sensor, the second sensor being a biometric sensor; and
at least one memory storing computer-readable instructions that, when executed by at least one processor, cause the system to:
receive first data representing intracranial waveforms generated by the first sensor at a first location communicatively coupled to a skull of a patient;
receive second data representing a non-cranial biophysical characteristic detected by the second sensor communicatively coupled to a peripheral neuropathy of the patient;
determine an intracranial pulsatile state of the patient based on the first data and the second data;
detect an abnormal neurological characteristic based on the intracranial pulsatile state; and
cause an output representing the abnormal neurological characteristic to be presented.

15. The system of claim 14,
wherein,
the computer-readable instructions, when executed by the one or more processor, further cause the system to:
identify, using the first data, an indication of a perturbation of the patient in at least one of a first frequency band corresponding to respiratory activity or a second frequency band corresponding to cardiac activity, detecting of the abnormal neurological characteristic is based at least partially on the indication of the perturbation.

16. The system of claim 15, further comprising:
an abdominal binder,
wherein,
the perturbation includes a change of abdominal constriction of the patient using the abdominal binder.

17. The system of claim 15,
wherein,
the perturbation includes a change between a standing position and a laying or siting position.

18. The system of claim 15,
wherein,
the perturbation includes a change between a regular breathing pattern and Valsalva breathing.

19. The system of claim 14, further comprising:
a third sensor, the third sensor being an accelerometer,
wherein,
the computer-readable instructions, when executed by one or more processor, further cause the system to:
receive accelerometer data generated by the accelerometer at third location on the patient; and
determine a head position based on the accelerometer data,
the intracranial pulsatile state includes a correlation with the head position.

20. The system of claim 14,
wherein,
detecting of the abnormal neurological characteristic includes:
determining a first brain pulsation signature based on cerebral hemodynamics represented by the intracranial waveforms correlated with the non-cranial biophysical characteristic; and
comparing the first brain pulsation signature to a second brain pulsation signature correlated with the non-cranial biophysical characteristic.
